(19) United States
(12) Patent Application Publication
(10) Pub. No.: US 2008/0249146 A1
(43) Pub. Date: Oct. 9, 2008

(54) Imidazole and Thioazole Derivatives as Antiviral Agents

(76) Inventors: Immacolata Conte, Pomezia (Rome) (IT); Jose Ignacio Martin Hernando, Pomezia (Rome) (IT); Savina Malancona, Pomezia (Rome) (IT); Jesus Maria Ontoria Ontoria, Pomezia (Rome) (IT); Ian Stansfield, Ariccia (Rome) (IT)

Correspondence Address: MERCK AND CO., INC, P O BOX 2000, RAHWAY, NJ, 07065-0907, US

(21) Appl. No.: 11/632,754

(22) PCT Filed: Jul. 14, 2005
(86) PCT No.: PCT/GB05/50111

§ 371 Date: Feb. 11, 2008

(30) Foreign Application Priority Data
Jul. 22, 2004 (GB) 0416396.0

(51) Int. Cl. A61K 31/429; A61K 31/4188; C07D 513/02; A61P 31/12; C07D 235/02

(52) U.S. Cl. 514/368; 514/393; 548/303.7; 548/154

(57) ABSTRACT

The present invention relates to compounds of the formula (I), wherein R¹, R², A, B, D, E, F, G and Ar are as defined herein, and pharmaceutically acceptable salts thereof, useful in the prevention and treatment of hepatitis C infections.

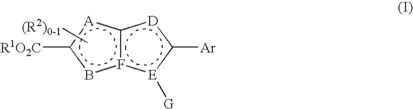

Imidazole and thiazole derivatives as antiviral agents This invention relates to compounds which can act as inhibitors of viral polymerases, especially the hepatitis C virus (HCV) polymerase, to uses of such compounds in the treatment and prevention of infection by hepatitis C virus, and to their preparation.

The hepatitis C virus (HCV) is the major causative agent of parenterally-transmitted and sporadic non-A, non-B hepatitis (NANB-H). Some 1% of the human population of the planet is believed to be affected. Infection by the virus can result in chronic hepatitis and cirrhosis of the liver, and may lead to hepatocellular carcinoma. Currently no vaccine nor established therapy exists, although partial success has been achieved in a minority of cases by treatment with recombinant interferon-(X, either alone or in combination with ribavirin. There is therefore a pressing need for new and broadly-effective therapeutics.

Several virally-encoded enzymes are putative targets for therapeutic intervention, including a metalloprotease (NS2-3), a serine protease (NS3), a helicase (NS3), and an RNA-dependent RNA polymerase (NS5B). Of these, the polymerase plays an essential role in replication of the virus and is therefore an important target in the fight against hepatitis C.

Certain imidazole and thiazole derivatives have been disclosed in the art but none are disclosed as being useful as inhibitors of hepatitis C virus (HCV) polymerase.

European Patent Application 0 407 102 A1 (Merck & Co., Inc.) discloses substituted imidazo-fused 5-membered ring heterocyclic compounds of general formula (A):

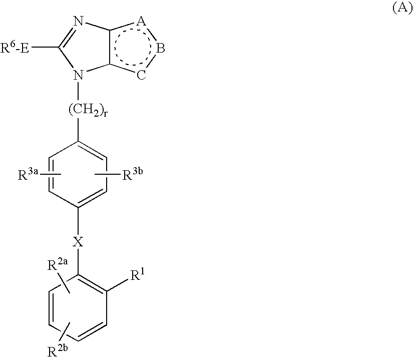

where R¹, R^{2a}, R^{2b}, R^{3a}, R^{3b}, R⁶, A, B, C, r and X are defined therein. These compounds are useful as angiotensin II antagonists.

European Patent Application 0 461 040 A1 (Roussel-Uclaf) discloses imidazole derivatives of general formula (B):

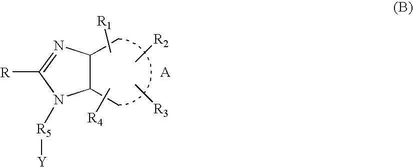

where A represents the rest of the carbocycle or heterocycle, and R, R¹, R², R³, R⁴, R⁵ and Y are defined therein. These compounds were tested for their angiotensin II antagonist activity.

Published International Application WO 98/37075 (Boehringer Ingelheim Pharma KG) discloses disubstituted heterocycles of the general formula (C):

Rₐ-A-Het-B—Ar-E (C)

where Het is a bicyclic heterocycle of formula:

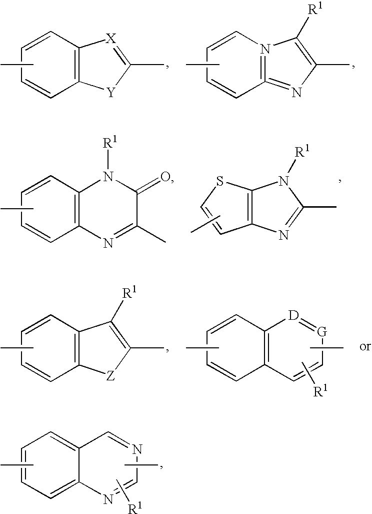

and Rₐ, A, B, E, Ar, R¹, D, G, X, Y and Z are defined therein. The compounds of formula (C) where E is a R_{b}NH—C(═NH)— group are disclosed as having pharmacological properties, in particular in inhibiting thrombin and prolonging thrombin time.

Published International Application WO99/00372 (Fujisawa Pharmaceutical Co., Ltd.) discloses sulfonamide compounds of general formula (D):

R¹—SO₂NHCO-A-X—R² (D)

where A represents an optionally substituted polyheterocyclic group except benzimidazolyl, indolyl, 4,7-dihydrobenzimidazolyl and 2,3-dihydrobenzoxazinyl, and R¹, R² and X are defined therein. These compounds are useful in treating diseases curable based on the hypoglycemic effect and diseases curable based on the cGMP-PDE inhibitory, leiolytic, bronchodilating, vasodilating, smooth muscle cell inhibitory and antiallergic effects.

However, none of these disclosures relate to the treatment of hepatitis C virus infections.

Khozeeva et al. (Zhurnal Organicheskoi Khimii, 1977, 13, 232) disclose the compound of the following structure:

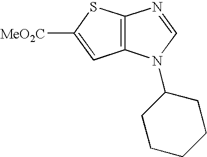

Abignente et al. (Farmaco, 1981, 36, 893) disclose the compound of the following structure and a process for its preparation:

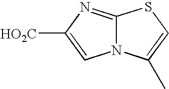

Robert et al. (European Journal of Medicinal Chemistry, 1975, 10, 59) disclose the compound of the following structure and a process for its preparation:

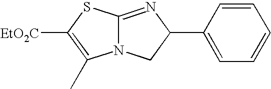

Again, none of these disclosures relate to the treatment of hepatitis C virus infections.

It has now surprisingly been found that certain imidazole and thiazole derivatives, including certain of the known compounds referred to above, act as inhibitors of the hepatitis C virus (HCV) polymerase enzyme.

Thus, in one aspect, there is provided the use of a compound of formula (I):

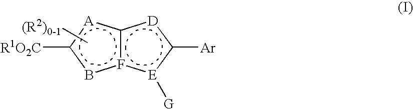

or a pharmaceutically acceptable salt thereof, for the manufacture of a medicament for the treatment or prevention of infection by hepatitis C virus,
wherein

A, B and D are each C, N, O or S;

E and F are C or N;

the dotted circle within the five-membered ring indicates that the ring may be unsaturated or partially saturated;

R¹ is hydrogen or C₁₋₆ alkyl;

R² is halogen, hydroxy, C₁₋₆ alkyl, C₂₋₆ alkenyl, C₁₋₆ alkoxy or aryl;

G is hydrogen, C₁₋₆ alkyl, C₂₋₆ alkenyl, where said C₁₋₆ alkyl and C₂₋₆ alkenyl groups are optionally substituted by C₁₋₄ alkoxy or up to 5 fluorine atoms, or a non-aromatic ring of 3 to 8 ring atoms where said ring may contain a double bond and/or may contain an O, S, SO, SO₂ or NH moiety and where said ring is optionally substituted by methyl, ethyl or fluorine, or aryl;

Ar is a moiety containing at least one aromatic ring and possesses 5-, 6-, 9- or 10-ring atoms optionally containing 1, 2 or 3 heteroatoms independently selected from N, O and S.

In one embodiment of the present invention, A, B and D are C, N or S. Preferably, A is S when B is C, and A is C when B is S. Preferably, D is N.

In another embodiment, both five-membered rings are unsaturated.

In another embodiment, R¹ is hydrogen or C₁₋₄ alkyl. Preferably, R¹ is hydrogen, methyl or ethyl. More preferably, R¹ is hydrogen.

In another embodiment, R² is C₁₋₆ alkyl, C₁₋₆ alkoxy or aryl. Preferably, R² is C₁₋₄ alkyl or aryl. More preferably, R² is methyl or phenyl. Preferably, R² is absent.

In another embodiment, G is hydrogen, C₃₋₈ cycloalkyl, C₃₋₈ cycloalkenyl or aryl. Preferably, G is hydrogen, cyclohexyl, cyclohexenyl or phenyl. More preferably, G is cyclohexyl or cyclohexenyl.

In another embodiment, Ar is a 5- or 6-membered aromatic ring, optionally containing 1, 2 or 3 heteroatoms independently selected from N, O and S. Preferably, Ar is a 6-membered ring containing 0, 1 or 2 N atoms, such as phenyl, 1-pyridyl, 2-pyridyl, 3-pyridyl, pyridazinyl, pyrimidinyl and pyrazinyl. More preferably, Ar is phenyl.

In a further embodiment of the present invention, there is provided the use of a compound of formula (Ia):

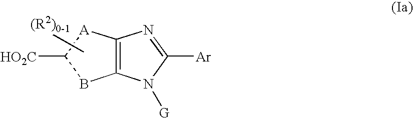

or a pharmaceutically acceptable salt thereof, for the manufacture of a medicament for the treatment or prevention of infection by hepatitis C virus, wherein A, B, R², G and Ar are as defined in relation to formula (I), and the dotted line represents a single or double bond.

Preferably, A is S when B is C and A is C when B is S. More preferably, A is C and B is S.

Preferably, R² is C₁₋₆ alkyl or aryl. More preferably, R² is methyl or phenyl. Preferably, R² is absent.

Preferably, G is hydrogen, C₃₋₈ cycloalkyl, C₃₋₈ cycloalkenyl or aryl. More preferably, G is cyclohexyl or cyclohexenyl. More preferably, G is cyclohexyl.

Preferably, Ar is a 6-membered ring containing 0, 1 or 2 N atoms. More preferably, Ar is phenyl.

In a further embodiment of the present invention, there is provided the use of a compound of formula (Ib):

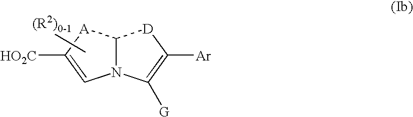

or a pharmaceutically acceptable salt thereof, for the manufacture of a medicament for the treatment or prevention of infection by hepatitis C virus, wherein A, D, R², G and Ar are as defined in relation to formula (I), and the dotted line represents a single or double bond.

Preferably, A is S when D is N and A is N when D is S. More preferably, A is S and D is N.

Preferably, R² is C₁₋₆ alkyl or aryl. More preferably, R² is methyl or phenyl. Preferably, R² is absent.

Preferably, G is hydrogen, C₃₋₈ cycloalkyl, C₃₋₈ cycloalkenyl or aryl.

Preferably, Ar is a 6-membered ring containing 0, 1 or 2 N atoms. More preferably, Ar is phenyl.

When any variable occurs more than one time in formula (I) or in any substituent, its definition on each occurrence is independent of its definition at every other occurrence.

When used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy.

The cycloalkyl groups referred to herein may represent, for example, cyclopropyl, cyclobutyl, cyclopentyl or cyclohexyl.

When used herein, the term “alkenyl” as a group or part of a group means that the group is straight or branched. Examples of suitable alkenyl groups include vinyl and allyl.

The cycloalkenyl groups referred to herein may represent, for example 1- or 2-cyclobutenyl, 1-, 2- or 3-cyclopentenyl or 1-, 2- or 3-cyclohexenyl.

When used herein, the term “aryl” as a group or part of a group means a carbocyclic aromatic ring. Examples of suitable aryl groups include phenyl and naphthyl.

When used herein, the term “halogen” means fluorine, chlorine, bromine and iodine. Preferred halogens are fluorine and chlorine.

Where a compound or group is described as “optionally substituted” one or more substituents may be present. Optional substituents may be attached to the compounds or groups which they substitute in a variety of ways, either directly or through a connecting group of which the following are examples: amine, amide, ester, ether, thioether, sulfonamide, sulfamide, sulfoxide, urea, thiourea and urethane. As appropriate an optional substituent may itself be substituted by another substituent, the latter being connected directly to the former or through a connecting group such as those exemplified above.

Specific compounds within the scope of this invention include:
1-cyclohexyl-2-phenyl-1H-thieno[3,2-d]imidazole-5-carboxylic acid,
3-cyclohexyl-2-phenyl-3H-thieno[2,3-d]imidazole-5-carboxylic acid,
3-cyclohexyl-6-methyl-2-phenyl-3-thieno[2,3-d]imidazole-5-carboxylic acid,
3-cyclohexyl-2,6-diphenyl-3H-thieno[2,3-d]imidazole-5-carboxylic acid,
5,6-diphenylimidazo[2,1-b][1,3]thiazole-2-carboxylic acid,
6-phenylimidazo[2,1-b]thiazole-2-carboxylic acid,
5-cyclohex-1-en-1-yl-6-phenylimidazo[2,1-b][1,3]thiazole-2-carboxylic acid,
3-cyclohex-1-en-1-yl-2-phenylimidazo[2,1-b][1,3]thiazole-6-carboxylic acid;
   and pharmaceutically acceptable salts thereof.

For use in medicine, the salts of the compounds of formula (I) will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulfonic acid, maleic acid, succinic acid, acetic acid, citric acid, tartaric acid, carbonic acid, phosphoric acid or sulfuric acid. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The salts may be formed by conventional means, such as by reacting the free base form of the product with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention includes within its scope prodrugs of the compounds of formula (I) above. In general, such prodrugs will be functional derivatives of the compounds of formula (I) which are readily convertible in vivo into the required compound of formula (I). Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulfate ester, or reduction or oxidation of a susceptible functionality.

The present invention includes within its scope solvates of the compounds of formula (I) and salts thereof, for example, hydrates.

The present invention also includes within its scope any enantiomers, diastereomers, geometric isomers and tautomers of the compounds of formula (I). It is to be understood that all such isomers and mixtures thereof are encompassed within the scope of the invention.

In another aspect of the invention, there is provided a method of inhibiting hepatitis C virus polymerase and/or of treating or preventing an illness due to hepatitis C virus, the method involving administering to a human or animal (preferably mammalian) subject suffering from the condition a therapeutically or prophylactically effective amount of the pharmaceutical composition described above or of a compound of formula (I), (Ia) or (Ib) as defined above, or a pharmaceutically acceptable salt thereof. “Effective amount” means an amount sufficient to cause a benefit to the subject or at least to cause a change in the subject's condition.

In a further embodiment of the present invention, there is provided the use of a compound of formula (I), (Ia) or (Ib), or a pharmaceutically acceptable salt thereof, for the manufacture of a medicament for the treatment or prevention of infection by hepatitis C virus, in combination with one or more other agents for the treatment of viral infections such as an antiviral agent, and/or an immunomodulatory agent such as α-, β- or γ-interferon, particularly α-interferon. Suitable antiviral agents include ribavirin and inhibitors of hepatitis C virus (HCV) polymerase, such as inhibitors of metalloprotease (NS2-3), serine protease (NS3), helicase (NS3) and RNA-dependent RNA polymerase (NS5B).

A further aspect of the invention provides a pharmaceutical composition comprising
1-cyclohexyl-2-phenyl-1H-thieno[3,2-d]imidazole-5-carboxylic acid,
3-cyclohexyl-2-phenyl-3H-thieno[2,3-d]imidazole-5-carboxylic acid,
3-cyclohexyl-6-methyl-2-phenyl-3-thieno[2,3-d]imidazole-5-carboxylic acid,
3-cyclohexyl-2,6-diphenyl-3H-thieno[2,3-d]imidazole-5-carboxylic acid,
5,6-diphenylimidazo[2,1-b][1,3]thiazole-2-carboxylic acid,
6-phenylimidazo[2,1-b]thiazole-2-carboxylic acid,
5-cyclohex-1-en-1-yl-6-phenylimidazo[2,1-b][1,3]thiazole-2-carboxylic acid, or
3-cyclohex-1-en-1-yl-2-phenylimidazo[2,1-b][1,3]thiazole-6-carboxylic acid;
   or a pharmaceutically acceptable salt thereof, in association with a pharmaceutically acceptable carrier. The composition may be in any suitable form, depending on the intended method of administration. It may for example be in the form of a tablet, capsule or liquid for oral administration, or of a solution or suspension for administration parenterally. The composition may be prepared by admixing at least one active ingredient, or a pharmaceutically acceptable salt thereof, with one or more pharmaceutically acceptable adjuvants, diluents or carriers and/or with one or more other therapeutically or prophylactically active agents.

A further aspect of the invention provides 1-cyclohexyl-2-phenyl-1H-thieno[3,2-d]imidazole-5-carboxylic acid,
3-cyclohexyl-2-phenyl-3H-thieno[2,3-d]imidazole-5-carboxylic acid,
3-cyclohexyl-6-methyl-2-phenyl-3-thieno[2,3-d]imidazole-5-carboxylic acid,
3-cyclohexyl-2,6-diphenyl-3H-thieno[2,3-d]imidazole-5-carboxylic acid,
5,6-diphenylimidazo[2,1-b][1,3]thiazole-2-carboxylic acid,
6-phenylimidazo[2,1-b]thiazole-2-carboxylic acid,
5-cyclohex-1-en-1-yl-6-phenylimidazo[2,1-b][1,3]thiazole-2-carboxylic acid,
3-cyclohex-1-en-1-yl-2-phenylimidazo[2,1-b][1,3]thiazole-6-carboxylic acid;
   or a pharmaceutically acceptable salt thereof for use in therapy.

A further aspect of the invention provides 1-cyclohexyl-2-phenyl-1H-thieno[3,2-d]imidazole-5-carboxylic acid,
3-cyclohexyl-2-phenyl-3H-thieno[2,3-d]imidazole-5-carboxylic acid,
3-cyclohexyl-6-methyl-2-phenyl-3-thieno[2,3-d]imidazole-5-carboxylic acid,
3-cyclohexyl-2,6-diphenyl-3H-thieno[2,3-d]imidazole-5-carboxylic acid,
5,6-diphenylimidazo[2,1-b][1,3]thiazole-2-carboxylic acid,
6-phenylimidazo[2,1-b]thiazole-2-carboxylic acid,
5-cyclohex-1-en-1-yl-6-phenylimidazo[2,1-b][1,3]thiazole-2-carboxylic acid,
3-cyclohex-1-en-1-yl-2-phenylimidazo[2,1-b][1,3]thiazole-6-carboxylic acid;
   or a pharmaceutically acceptable salt thereof.

The dosage rate at which the compound is administered will depend on a variety of factors including the activity of the specific compound employed, the metabolic stability and length of action of that compound, the age of the patient, body weight, general health, sex, diet, mode and time of administration, rate of excretion, drug combination, the severity of the particular condition and the host undergoing therapy. For the treatment or prevention of infection by hepatitis C virus, suitable dosage levels may be of the order of 0.02 to 5 or 10 g per day, with oral dosages two to five times higher. For instance, administration of from 10 to 50 mg of the compound per kg of body weight from one to three times per day may be in order. Appropriate values are selectable by routine testing. The compound may be administered alone or in combination with other treatments, either simultaneously or sequentially. For instance, it may be administered in combination with effective amounts of antiviral agents, immunomodulators, anti-infectives or vaccines known to those of ordinary skill in the art. It may be administered by any suitable route, including orally, intravenously, cutaneously and subcutaneously. It may be administered directly to a suitable site or in a manner in which it targets a particular site, such as a certain type of cell. Suitable targeting methods are already known.

Compounds of general formula (I), (Ia) or (Ib) may be prepared by methods disclosed in the documents hereinbefore referred to and by methods known in the art of organic synthesis as set forth below.

According to a general process (A), compounds of formula (I), where A is S, B and F are C, D and E are N, and R² is absent, may be prepared by reacting a compound of formula (II):

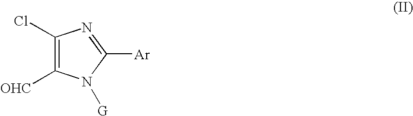

where Ar and G are as defined for formula (I), with methyl thioglycolate. The reaction is conveniently performed in the presence of a base, such as sodium ethoxide, in a suitable solvent, such as ethanol.

The compound of formula (II) may be formed by the reaction of a compound of formula (III):

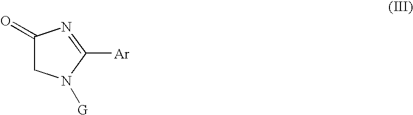

where Ar and G are as defined for formula (I), with the Vilsmeier-Haack reagent preformed from phosphorus oxychloride and DMF.

Where it is not commercially available, the starting material of formula (III) may be prepared by methods analogous to those described in the accompanying Examples, or by standard methods well known from the art.

It will be understood that any compound of formula (I) initially obtained from any of the above processes may, where appropriate, subsequently be elaborated into a further compound of formula (I) by techniques known from the art.

By way of specific example, a compound of formula (I) wherein R¹ represents hydrogen may be converted into the corresponding compound wherein R¹ is other than hydrogen by means of conventional esterification procedures, e.g. by treatment with the appropriate alcohol of formula R¹—OH in the presence of a mineral acid such as hydrochloric acid. A compound of formula (I) wherein R¹ is other than hydrogen may be converted into the corresponding compound wherein R¹ is hydrogen by means of standard saponification techniques, e.g. by treatment with an alkaline reagent such as sodium hydroxide or lithium hydroxide.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3rd edition, 1999. The protecting groups may be removed at a convenient subsequent stage using methods known from the art.

The following Examples are illustrative of the invention.

The compounds of the invention were tested for inhibitory activity against the HCV RNA dependent RNA polymerase (NS5B) in an enzyme inhibition assay (example i) and generally have IC50's below 50 μM.

### i) In-vitro HCV NS5B Enzyme Inhibition Assay

WO 96/37619 describes the production of recombinant HCV RdRp from insect cells infected with recombinant baculovirus encoding the enzyme. The purified enzyme was shown to possess in vitro RNA polymerase activity using RNA as template. The reference describes a polymerisation assay using poly(A) and oligo(U) as a primer or an heteropolymeric template. Incorporation of tritiated UTP or NTPs is quantified by measuring acid-insoluble radioactivity. The present inventors have employed this assay to screen the various compounds described above as inhibitors of HCV RdRp.

Incorporation of radioactive UMP was measured as follows. The standard reaction (50 μl) was carried out in a buffer containing 20 mM tris/HCl pH 7.5, 5 mM MgCl₂, 1 mM DTT, 50 mM NaCl, 0.03% N-octylglucoside, 1 μCi [³H]-UTP (40 Ci/mmol, NEN), 10 μM UTP and 10 μg/ml poly(A) or 5 μM NTPs and 5 μg/ml heteropolymeric template. Oligo(U)₁₂ (1 μg/ml, Genset) was added as a primer in the assay working on Poly(A) template. The final NS5B enzyme concentration was 5 nM. The order of assembly was: 1) compound, 2) enzyme, 3) template/primer, 4) NTP. After 1 h incubation at 22° C. the reaction was stopped by adding 50 μl of 20% TCA and applying samples to DE81 filters. The filters were washed thoroughly with 5% TCA containing 1M Na₂HPO₄/NaH₂PO₄, pH 7.0, rinsed with water and then ethanol, air dried, and the filter-bound radioactivity was measured in the scintillation counter. Carrying out this reaction in the presence of various concentrations of each compound set out above allowed determination of IC₅₀ values by utilising the formula:

% Residual activity=100/(1+[I]/IC₅₀)S

where [I] is the inhibitor concentration and “s” is the slope of the inhibition curve.

### ii) General Procedures

All solvents were obtained from commercial sources (Fluka, puriss.) and were used without further purification. With the exception of routine deprotection and coupling steps, reactions were carried out under an atmosphere of nitrogen in oven dried (110° C.) glassware. Organic extracts were dried over sodium sulfate, and were concentrated (after filtration of the drying agent) on rotary evaporators operating under reduced pressure. Flash chromatography was carried out on silica gel following published procedure (W. C. Still et al., J. Org. Chem. 1978, 43, 2923) or on semi-automated flash chromatography systems utilising pre-packed columns.

Reagents were usually obtained directly from commercial suppliers (and used as supplied) but a limited number of compounds from in-house corporate collections were utilised. In the latter case the reagents are readily accessible using routine synthetic steps that are either reported in the scientific literature or are known to those skilled in the art.

¹H nmr spectra were recorded on Bruker AM series spectrometers operating at (reported) frequencies between 300 and 600 MHz. Chemical shifts (6) for signals corresponding to non-exchangeable protons (and exchangeable protons where visible) are recorded in parts per million (ppm) relative to tetramethylsilane and are measured using the residual solvent peak as reference. Signals are tabulated in the order: multiplicity (s, singlet; d, doublet; t, triplet; q, quartet; m, multiplet; br, broad, and combinations thereof); coupling constant(s) in hertz; number of protons. Mass spectral (MS) data were obtained on a Perkin Elmer API 100 operating in negative (ES⁻) or positive (ES⁺) ionization mode and results are reported as the ratio of mass over charge (m/z) for the parent ion only. Preparative scale HPLC separations were carried out on a Waters Delta Prep 4000 separation module, equipped with a Waters 486 absorption detector or on a Gilson preparative system. In all cases compounds were eluted with linear gradients of water and acetonitrile both containing 0.1% TFA using flow rates between 15 and 25 mL/min.

The following abbreviations are used in the examples:

DMF: dimethylformamide; DCM: dichloromethane; DMSO: dimethylsulfoxide; TFA: trifluoroacetic acid; THF: tetrahydrofuran; MeOH: methanol; AcOEt: ethyl acetate; MeCN: acetonitrile; Et₂O: ethyl ether; CHCl₃: chloroform; CDCl₃: chloroform-d; CD₃OD: methyl-d₃ alcohol-d; HCl: hydrogen chloride; NaOH: sodium hydroxide; NaHCO₃: sodium hydrogencarbonate; NH₄Cl: ammonium chloride; NH₄OH: ammonium hydroxide; min: minutes; h: hour(s); eq.: equivalent(s); RT: room temperature; MS: molecular sieves; RP-HPLC: reversed phase high-pressure liquid chromatography; Et₃N: triethylamine; NH₃: ammonia; AcOH: acetic acid; NaN₃: sodium azide; PPh₃: triphenylphosphine; TPAP: tetrapropylammonium perruthenate; MeMgBr: methylmagnesium bromide; NBS: N-bromosuccinimide; NaOMe: sodium methoxide; NMO: 4-methylmorpholine N-oxide; Br₂: bromine.

### EXAMPLE 1

### 3-cyclohexyl-2-phenyl-3H-thieno[3,2-d]imidazole-5-carboxylic acid

### Step 1: ethyl N-cyclohexylglycinate

A solution (0.22 M) of glycine ethyl ester hydrochloride in ethanol was treated with sodium acetate (1.5 eq.) and sodium cyanoborohydride (1.6 eq.). Cyclohexanone (1 eq.) was added dropwise to the resulting suspension. The reaction mixture was stirred at RT overnight, then was acidified to pH 2 with aqueous HCl (6 N). The solvent was evaporated and the residue diluted with water. The aqueous phase was extracted with Et₂O and then aqueous NaOH (2 N) was added to the aqueous phase until basic pH. The resulting aqueous phase was extracted with Et₂O and the combined organic phases were washed with brine then dried and concentrated to afford the title compound (89%) as an oil. ¹H NMR (400 MHz, DMSO-d₆, 300 K) δ 0.9-1.0 (m, 2H), 1.1-1.2 (m, 5H), 1.5-1.8 (m, 6H), 2.1 (bs, 1H), 2.3-2.4 (m, 1H), 3.3 (s, 2H), 4.08 (q, J=7.2 Hz, 2H); MS (ES⁺) m/z 186 (M+H)⁺.

### Step 2: A2-cyclohexylglycinamide

A methanolic solution of NH₃ (2 M, 5 eq.) was added to a pressure vessel containing ethyl N-cyclohexylglycinate (from Step 1). The vessel was closed and the reaction mixture was heated at 100° C. overnight. Subsequent evaporation of the solvent, followed by trituration with petroleum ether and filtration gave the title compound (73%) as a solid. ¹H NMR (400 MHz, DMSO-d₆, 300 K) δ 0.9-1.2 (m, 5H), 1.5-1.8 (m, 5H), 2.2-2.3 (m, 1H), 3.0 (s, 2H), 3.3-3.4 (m, 1H), 7.0 (bs, 1H), 7.2 (bs, 1H); MS (ES⁺) m/z 157 (M+H)⁺.

### Step 3: 1-cyclohexyl-2-phenyl-1,5-dihydro-4H-imidazol-4-one

N²-cyclohexylglycinamide (from Step 2) was treated with triethylorthobenzoate (1.03 eq.) and catalytic amount of glacial AcOH. The reaction mixture was heated at 120° C. for 1.5 h then cooled to RT and concentrated. Acetone was added to the residue and the resulting precipitate was filtered to afford the title compound (52%) as a solid. ¹H NMR (300 MHz, CDCl₃, 300 K) δ 1.1-1.3 (m, 3H), 1.5-1.9 (m, 7H), 3.8-3.9 (m, 1H), 4.0 (s, 2H), 7.5-7.6 (m, 5H); MS (ES⁺) m/z 243 (M+H)⁺.

### Step 4: 4-chloro-1-cyclohexyl-2-phenyl-1H-imidazole-5-carbaldehyde

Phosphorus oxychloride (7 eq.) was added to ice-cold DMF (3 eq.). The mixture was left to reach RT, then 1-cyclohexyl-2-phenyl-1,5-dihydro-4H-imidazol-4-one (from Step 3) was added to the preformed Vilsmeier-Haack reagent. The mixture was heated to reflux for 1 h, then cooled to RT and poured into ice-cold water. The light yellow precipitate formed was filtered and washed with water and dried to afford the title compound (82%) as a solid. ¹H NMR (400 MHz, CDCl₃, 300 K) δ 1.1-1.4 (m, 3H), 1.6-1.8 (m, 5H), 2.3-2.5 (m, 2H), 4.2-4.3 (m, 1H), 7.5 (m, 5H), 9.8 (s, 1H); MS (ES⁺) m/z 289 (M+H)⁺.

### Step 5: ethyl 1-cyclohexyl-2-phenyl-1H-thieno[3,2-d]imidazole-5-carboxylate

A solution (1.15 M, 1.2 eq.) of methyl thioglycolate in ethanol was added dropwise to a stirred solution (0.17 M, 1.2 eq.) of sodium in ethanol at RT. 4-chloro-1-cyclohexyl-2-phenyl-1H-imidazole-5-carbaldehyde (from Step 4) was then added portionwise over 15 min and the reaction mixture was heated to reflux for 18 h. After cooling down, the mixture was concentrated and the residue was diluted with DCM and water. The aqueous phase was separated and extracted with DCM. The combined organic phases were washed with brine then dried and concentrated. The crude was purified by flash chromatography on silica gel (1:9 AcOEt/petroleum ether) to afford the title compound (31%) as a solid. ¹H NMR (400 MHz, CDCl₃, 300 K) δ 1.2-1.4 (m, 6H), 1.7-2.1 (m, 7H), 4.2-4.3 (m, 1H), 4.40 (q, J 8 Hz, 2H), 7.5-7.6 (m, 5H), 7.8 (s, 1H); MS (ES⁺) m/z 355 (M+H)⁺.

### Step 6: 1-cyclohexyl-2-phenyl-1H-thieno[3,2-d]imidazole-5-carboxylic acid

A solution (0.25 M) of ethyl 1-cyclohexyl-2-phenyl-1H-thieno[3,2-d]imidazole-5-carboxylate (from Step 5) in MeOH/THF (1:1) was treated with aqueous NaOH (1 N solution, 2 eq.) and the reaction stirred at RT for 4 h. The reaction mixture was concentrated and acidified with aqueous HCl (1 N). The resulting precipitate was collected by filtration and purified by RP-HPLC (Conditions: Waters X-TERRA MS C18, 5 micron, 19×100 mm; flow: 20 mL/min; Gradient: A: H₂O+0.1% TFA; B: MeCN+0.1% TFA; 80% A isocratic for 2 min, linear to 40% A in 7 min, isocratic for 1 min, linear to 20% A in 2 min, isocratic for 1 min then linear in 1 min to 0% A) to afford the title compound (40%) as a solid. ¹H NMR (300 MHz, DMSO-d₆+TFA, 300 K) δ 1.2-1.4 (m, 3H), 1.5-1.6 (m, 1H), 1.8-2.1 (m, 6H), 4.3 (m, 1H), 7.5-7.6 (m, 5H), 8.0 (s, 1H); MS (ES⁺) m/z 327 (M+H)⁺.

### EXAMPLE 2

### 3-cyclohexyl-2-phenyl-3H-thieno[2,3-d]imidazole-5-carboxylic acid

### Step 1: N-cyclohexylbenzamide

A solution (0.47 M) of cyclohexylamine in DCM was added dropwise to a stirred solution (0.14 M) of benzylchloride (1.1 eq.) and Et₃N (1.5 eq.) in DCM at 0° C. The reaction mixture was stirred for 0.5 h at RT, then the solvents were evaporated and the residue was dissolved in AcOEt. The organic layer was washed sequentially with aqueous HCl (1 N), aqueous NaHCO₃ (saturated solution) and brine then dried and evaporated giving the title compound (95%) that was used as such in next reaction. ¹H NMR (400 MHz, CDCl₃, 300 K) δ 1.1-1.3 (m, 3H), 1.3-1.5 (m, 2H), 1.6-1.7 (m, 1H), 1.7-1.8 (m, 2H), 2.0-2.1 (m, 2H), 3.9-4.0 (m, 1H), 6.1 (bs, 1H), 7.3-7.4 (m, 2H), 7.4-7.5 (m, 1H), 7.7-7.8 (m, 2H); MS (ES⁺) m/z 204 (M+H)⁺.

### Step 2: N-(azidoacetyl)-N-cyclohexylbenzamide

A solution (0.2 M) of N-cyclohexylbenzamide (from Step 1) in toluene was treated with chloroacetyl chloride (1.1 eq.). The reaction mixture was refluxed for 3 h then the solvents were evaporated. The residue was dissolved in DMSO, and to this solution (0.2 M) NaN₃ (3 eq.) was added. The reaction mixture was stirred at RT for 1 h, then it was poured onto water and extracted with Et₂O. The organic layer was washed with brine then dried and evaporated. The crude was purified by flash chromatography on silica gel (AcOEt/petroleum ether, from 10% AcOEt to 20% AcOEt) to afford the title compound (64%) as a solid. ¹H NMR (400 MHz, CDCl₃, 300 K) δ 1.1-1.3 (m, 3H), 1.5-1.6 (m, 1H), 1.8-1.9 (m, 4H), 2.1-2.2 (m, 2H), 3.8 (s, 2H), 4.0-4.1 (m, 1H), 7.4-7.5 (m, 2H), 7.6-7.7 (m, 1H), 7.7-7.8 (m, 2H); MS (ES⁺) m/z 309 (M+Na)⁺, 287 (M+H)⁺.

### Step 3: 3-cyclohexyl-2-phenyl-3,5-dihydro-4H-imidazol-4-one

A solution (0.06 M) of N-(azidoacetyl)-N-cyclohexylbenzamide (from Step 2) in toluene was treated with PPh₃ (1.1 eq.). The reaction mixture was stirred at RT for 4.5 h then solvent was evaporated and the crude was purified by flash chromatography on silica gel (1:1 AcOEt/petroleum ether) to give the title compound (77%) as a solid. ¹H NMR (400 MHz, CDCl₃, 300 K) δ 1.1-1.3 (m, 3H), 1.5-1.6 (m, 1H), 1.6-1.7 (m, 2H), 1.7-1.8 (m, 2H), 2.2-2.3 (m, 2H), 3.5-3.6 (m, 1H), 4.2 (s, 2H), 7.5-7.6 (m, 5H); MS (ES⁺) m/z 243 (M+H)⁺.

### Step 4: 5-chloro-1-cyclohexyl-2-phenyl-1H-imidazole-4-carbaldehyde

Phosphorus oxychloride (3 eq.) was added dropwise to a solution (0.95 M, 4 eq.) of DMF in CHCl₃ at 0° C. The reaction mixture was allowed to warm to RT then a solution (0.16 M) of 3-cyclohexyl-2-phenyl-3,5-dihydro-4H-imidazol-4-one (from Step 3) in CHCl₃ was added. The reaction mixture was heated to reflux for 2 h then solvents were evaporated. The residue was dissolved in phosphorus oxychloride, and the resulting solution (0.12 M) was refluxed for 18 h. Then it was concentrated and the residue diluted with AcOEt and water. The mixture was stirred for 15 min at RT, then it was neutralized to pH 7 with aqueous NaOH (2 N). The layers were separated and the aqueous layer was extracted with AcOEt. The combined organic layers were washed with brine, dried and evaporated to give a crude that was purified by flash chromatography on silica gel (AcOEt/petroleum ether, from 15% AcOEt to 20% AcOEt) to afford the title compound (56%) as a solid. ¹H NMR (400 MHz, CDCl₃, 300 K) δ 1.2-1.3 (m, 3H), 1.6-1.7 (m, 1H), 1.8-1.9 (m, 4H), 2.2-2.3 (m, 2H), 4.1-4.3 (m, 1H), 7.5-7.6 (m, 5H), 10.0 (s, 1H); MS (ES⁺) m/z 289 (M+H)⁺.

### Step 5: 3-cyclohexyl-2-phenyl-3H-thieno[2,3-d]imidazole-5-carboxylic acid

Methyl thioglycolate (1.5 eq.) was added to a solution (1.44 M, 4 eq.) of NaOMe in MeOH at RT, then a solution (0.12 M) of 5-chloro-1-cyclohexyl-2-phenyl-1H-imidazole-4-carbaldehyde (from Step 4) in MeOH was added. The reaction mixture was refluxed for 2 h. After evaporation of the solvent, the residue was diluted with AcOEt and aqueous NH₄Cl (saturated solution) was added, the layers were separated and the aqueous layer was extracted with AcOEt. The combined organic layers were dried and evaporated to give a residue that was dissolved in THF, and the resulting solution (0.24 M) was treated with aqueous NaOH (1 N, 4.2 eq.). The reaction mixture was heated at 60° C. for 2 h. Solvents were evaporated and AcOEt was added. The organic layer was washed twice with aqueous NaOH (1 N) and the combined aqueous layers were acidified to pH 1 with aqueous HCl (6 N). The acidic aqueous layer was extracted with AcOEt, and the combined organic layers were dried and evaporated giving a crude that was purified by flash chromatography on silica gel (MeOH/DCM, from 5% MeOH to 15% MeOH) to afford the title compound (32%) as a solid. ¹H NMR (300 MHz, DMSO-d₆, 300 K) δ 1.2-1.3 (m, 3H), 1.5-1.7 (m, 1H), 1.8-2.0 (m, 6H), 4.2-4.3 (m, 1H), 7.5-7.6 (m, 5H), 7.7 (s, 1H); MS (ES⁺) m/z 327 (M+H)⁺.

### EXAMPLE 3

### 3-cyclohexyl-6-methyl-2-phenyl-3H-thieno[2,3-d]imidazole-5-carboxylic acid

### Step 1: 1-(5-chloro-1-cyclohexyl-2-phenyl-1H-imidazol-4-yl)ethanone

A solution (0.08 M) of 5-chloro-1-cyclohexyl-2-phenyl-1H-imidazole-4-carbaldehyde (from Example 2, Step 4) in Et₂O was treated with MeMgBr (3 M solution in Et₂₀, 1 eq.) at 0° C. The reaction mixture was stirred at 0° C. for 45 min then quenched with aqueous NH₄Cl (saturated solution) and extracted with AcOEt. The combined organic layers were dried and evaporated. The resulting residue was dissolved in CH₃CN and the solution (0.08 M) was treated with 4 Å MS, NMO (1.1 eq.) and TPAP (0.1 eq.). The reaction mixture was stirred at RT for 2 h. After evaporation of the solvent the crude was purified by flash chromatography on silica gel (7:1 AcOEt/petroleum ether) to afford the title compound (38%) as a solid. ¹H NMR (400 MHz, CDCl₃, 300 K) δ 1.2-1.3 (m, 3H), 1.6-1.7 (m, 1H), 1.8-1.9 (m, 4H), 2.3-2.4 (m, 2H), 2.6 (s, 3H), 4.1-4.3 (m, 1H), 7.5-7.6 (m, 5H); MS (ES⁺) m/z 303 (M+H)⁺.

### Step 2: 3-cyclohexyl-6-methyl-2-phenyl-3H-thieno[2,3-d]imidazole-5-carboxylic acid

Methyl thioglycolate (3.0 eq.) was added at RT to a solution (0.24 M, 8 eq.) of NaOMe in MeOH, then a solution (0.015 M) of 1-(5-chloro-1-cyclohexyl-2-phenyl-1H-imidazol-4-yl)ethanone (from Step 1) in MeOH was added. The reaction was refluxed for 20 h then solvent was evaporated and the resulting residue was dissolved in THF, and the solution (0.06 M) was treated with aqueous NaOH (1 N, 16 eq.) for 3.5 h at RT. Then, a small amount of MeOH and aqueous NaOH (1 N, 65 eq.) were added. The reaction mixture was heated at 60° C. for 5 h then it was concentrated and water was added. After acidification with TFA the crude was purified by RP-HPLC (Conditions: Waters X-TERRA MS C18, 10 micron, 19×150 mm; flow: 18 mL/min; Gradient: A: H₂O+0.05% TFA; B: MeCN+0.05% TFA; 80% A isocratic for 2 min, linear to 70% A in 2 min, linear to 60% A in 2 min, linear to 50% A in 2 min, isocratic for 6 min, linear to 45% A in 2 min, isocratic for 2 min then linear to 35% A in 1 min) to afford the title compound (15%) as a solid. ¹H NMR (300 MHz, CD₃OD, 300 K) δ 1.3-1.4 (m, 3H), 1.6-1.7 (m, 1H), 1.9-2.1 (m, 6H), 2.7 (s, 3H), 4.3-4.4 (m, 1H), 7.6-7.7 (m, 5H); MS (ES⁺) m/z 341 (M+H)⁺.

### EXAMPLE 4

### 6-phenylimidazo[2,1-b][1,3]thiazole-2-carboxylic acid

### Step 1: ethyl 2-amino-1,3-thiazole-5-carboxylate

A solution (1 M) of ethyl 3-ethoxyacrylate in water/dioxane (1:1) at −10° C. was treated with NBS (1.1 eq.). The reaction mixture was stirred at RT for 1 hour, then thiourea (1 eq.) was added and the reaction was heated at 80° C. for 1 h. After cooling at RT aqueous NH₄OH (saturated solution) was added. The resulting slurry was stirred at RT for 10 min and filtered. The resulting cake was washed with water and dried to afford the title compound (66%) as a pale yellow solid. ¹H NMR (400 MHz, DMSO, 300 K) δ 1.23 (t, J 6.8 Hz, 3H), 4.17 (q, J 6.8 Hz, 2H), 7.66 (s, 1H), 7.83 (s, 2H); MS (ES⁺) m/z 173 (M+H)⁺.

### Step 2: 6-phenylimidazo[2,1-b][1,3]thiazole-2-carboxylic acid

2-Bromo-1-phenylethanone (1 eq.) was added to a solution (0.2 M) of 2-amino-1,3-thiazole-5-carboxylate (from Step 1) in ethanol. The reaction mixture was heated to reflux overnight. After cooling down, the solvent was concentrated and the residue diluted with AcOEt. The organic phase was washed with water, brine then dried and evaporated. The residue was treated with Et₂O affording a crude that was dissolved in ethanol/water (3:1) and aqueous NaOH (1 N, 4 eq.) was added. The reaction mixture was refluxed for 3 hours. After cooling down, the mixture was acidified with aqueous HCl (3 N) and the resulting precipitate isolated by filtration, affording the title compound (26%) as a solid. ¹H NMR (400 MHz, DMSO, 300 K) δ 7.29 (t, J 7.3 Hz, 1H), 7.41 (t, J 7.5 Hz, 2H), 7.85 (d, J 7.2 Hz, 2H), 8.25 (s, 1H), 8.67 (s, 1H); MS (ES⁺) m/z 245 (M+H)⁺.

### EXAMPLE 5

### 5-cyclohex-1-en-1-yl-6-phenylimidazo[2,1-b][1,3]thiazole-2-carboxylic acid

Ethyl 6-phenylimidazo[2,1-b][1,3]thiazole-2-carboxylate was treated with acetic anhydride (4.25 eq.), glacial acetic acid (35 eq.), cyclohexanone (6 eq.) and 85% phosphoric acid (2.2 eq.). The reaction mixture was heated at 120° C. overnight. After cooling down, the reaction was treated with an ice-cold NH₄OH (saturated solution) and extracted with AcOEt. The combined organic layers were washed with aqueous HCl (1 N), aqueous NaHCO₃ (saturated solution) and brine then dried and evaporated. The crude was purified by flash chromatography on silica gel (1:5 AcOEt/petroleum ether) affording ethyl 5-cyclohex-1-en-1-yl-6-phenylimidazo[2,1-b][1,3]thiazole-2-carboxylate. The above compound was dissolved in ethanol and the resulting solution (0.5 M) treated with NaOH (1 N solution, 4 eq.). The mixture was heated at 80° C. for 1 h. After cooling it was acidified with aqueous HCl (3 N) and purified by HPLC (Conditions: Waters X-TERRA MS C18, 10 micron, 19×150 mm; flow: 18 mL/min; Gradient: A: H₂O+0.05% TFA; B: MeCN+0.05% TFA; 60% A isocratic for 2 min, linear to 20% A in 12 min) to afford the title compound (5%) as a solid. ¹H NMR (400 MHz, DMSO, 300 K) δ 1.7 (m, 4H), 2.2 (m, 4H), 6.11 (bs, 1H), 7.29 (t, J 7.3 Hz 1H), 7.42 (t, J 7.5 Hz, 2H), 7.71 (d, J 7.4 Hz, 2H); MS (ES⁺) m/z 325 (M+H)⁺.

### EXAMPLE 6

### 3-cyclohex-1-en-1-yl-2-phenylimidazo[2,1-b][1,3]thiazole-6-carboxylic acid

### Step 1: 1-cyclohexyl-2-phenylethanone

To a solution (0.25 M) of cyclohexanecarboxylic acid in DCM at 0° C. was added 1,1′-carbonyldiimidazole (1.5 eq.). The mixture was stirred at 0° C. for 1 h, then Et₃N (1.5 eq.) and N,O-dimethyhydroxylamine hydrochloride (1.3 eq.) were added. The reaction mixture was stirred at RT overnight and then concentrated. The resulting residue was diluted with AcOEt and washed with aqueous HCl (1 N), aqueous NaHCO₃ (saturated solution) and brine then dried and evaporated. This crude was dissolved in THF and the resulting solution (0.4 M) was cooled to −78° C. After dropwise addition of benzyl magnesium chloride (1 M solution in THF, 1.2 eq.) the reaction mixture was left to warm up to RT, then quenched with aqueous NH₄Cl (saturated solution), and extracted with AcOEt. The combined organic layers were washed with brine, dried and evaporated. The crude was purified by flash chromatography on silica gel (1:19 AcOEt/petroleum ether) to afford the title compound (54%) as colorless oil. ¹H NMR (400 MHz, DMSO, 300 K) δ 1.2 (m, 5H), 1.6 (m, 5H), 3.79 (s, 2H), 7.1-7.4 (m, 5H).

### Step 2: 4-cyclohex-1-en-1-yl-5-phenyl-1,3-thiazol-2-amine

A solution (0.1 M) of Br₂ (1.05 eq.) in dioxane was added dropwise to a solution (1 M) of 1-cyclohexyl-2-phenylethanone (from Step 1) in dioxane at RT. The reaction mixture was stirred for 30 min and then diluted with water. The aqueous phase was extracted with AcOEt and the combined organic layers were washed with water, aqueous NaHCO₃ (saturated solution) and brine then dried and evaporated. The crude was purified by flash chromatography on silica gel (1:25 AcOEt/petroleum ether) affording 2-bromo-1-cyclohexyl-2-phenylethanone. The above compound was dissolved in dioxane (0.5 M) and treated with thiourea (1.05 eq.). The reaction mixture was heated to reflux for 2 h then cooled and water (40 eq.) and aqueous NH₃ (33% solution, 3.75 eq.) were added and the resulting mixture was stirred at RT for 10 min. Then, the resulting precipitate was filtered affording the title compound (44%) as a solid. ¹H NMR (400 MHz, DMSO, 300 K) δ 1.5 (m, 4H), 1.9 (m, 2H), 2.1 (m, 2H), 5.7 (s, 1H), 6.9 (bs, 2H), 7.2 (m, 1H), 7.3 (m, 4H); MS (ES⁺) m/z 257 (M+H)⁺.

### Step 3: 3-cyclohex-1-en-1-yl-2-phenylimidazo[2,1-b][1,3]thiazole-6-carboxylic acid

A solution (0.2 M) of 4-cyclohex-1-en-1-yl-5-phenyl-1,3-thiazol-2-amine (from Step 2) in ethanol was treated with ethyl bromopyruvate (2 eq.) and Et₃N (2.2 eq.) were added. The reaction mixture was heated to reflux for 3 h then cooled and diluted with AcOEt. The organic solution was washed with water and brine then dried and evaporated. The crude was purified by flash chromatography on silica gel (1:5 AcOEt/petroleum ether) affording ethyl 3-cyclohex-1-en-1-yl-2-phenylimidazo[2,1-b][1,3]thiazole-6-carboxylate. The above compound was dissolved in ethanol (0.5 M) and treated with aqueous NaOH (1 N solution, 4 eq.). The reaction mixture was heated at 80° C. for 1 h, then cooled and acidified with aqueous HCl (3 N) and the resulting solid was filtered affording the title compound (24%) as a solid. ¹H NMR (400 MHz, DMSO, 300 K) δ 1.6 (m, 4H), 2.1 (m, 2H), 2.2 (m, 2H), 6.2 (bs, 1H), 7.5 (m, 5H), 8.2 (s, 1H); MS (ES⁺) m/z 325 (M+H)⁺.

The following table shows additional examples:

TABLE 1

| Example | Structure | MS (M + H)⁺ |
|---|---|---|
| 7 | 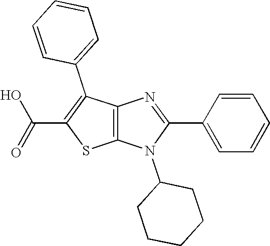 | 403 |
| 8 | 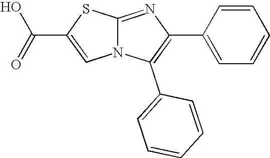 | 321 |

1. A method for treating or preventing infection by hepatitis C virus which comprises administering to a human or animal subject suffering from the infection a therapeutically or prophylactically effective amount of a compound of formula (I): 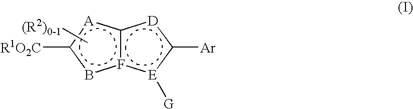
or a pharmaceutically acceptable salt thereof,
wherein
A, B and D are each C, N, O or S;
E and Fare C or N;
the dotted circle within the five-membered ring indicates that the ring may be unsaturated or partially saturated;
R¹ is hydrogen or C₁₋₆ alkyl;
R² is halogen, hydroxy, C₁₋₆ alkyl, C₂₋₆ alkenyl, C₁₋₆ alkoxy or aryl;
G is hydrogen, C₁₋₆ alkyl, C₂₋₆ alkenyl, where said C₁₋₆ alkyl and C₂₋₆ alkenyl groups are optionally substituted by C₁₋₄ alkoxy or up to 5 fluorine atoms, or a non-aromatic ring of 3 to 8 ring atoms where said ring may contain a double bond and/or may contain an O, S, SO, SO₂ or NH moiety and where said ring is optionally substituted by methyl, ethyl or fluorine, or aryl;
Ar is a moiety containing at least one aromatic ring and possesses 5-, 6-, 9- or 10-ring atoms optionally containing 1, 2 or 3 heteroatoms independently selected from N, O and S.

2. The method according to claim 1 wherein in the compound of formula (I) A, B and D are C, N or S.

3. The method according to claim 2 wherein in the compound of formula (I) A is S and B is C.

4. The method according to claim 2 wherein in the compound of formula (I) A is C and B is S.

5. The method according to claim 2 wherein in the compound of formula (I) (i) A is S, B is C, and D is N or (ii) A is C, B is S and D is N.

6. (canceled)

7. The method according to claim 1 wherein in the compound of formula (I) R¹ is hydrogen or C₁₋₄ alkyl.

8. (canceled)

9. The method according to claim 1 wherein in the compound of formula (I) R² is C₁₋₆ alkyl, C₁₋₆ alkoxy or aryl.

10. (canceled)

11. The method according to claim 1 wherein in the compound of formula (I) R² is absent.

12. The method according to claim 1 wherein in the compound of formula (I) G is hydrogen, C₃₋₈ cycloalkyl, C₃₋₈ cycloalkenyl or aryl.

13. (canceled)

14. The method according to claim 1 wherein in the compound of formula (I) Ar is a 5- or 6-membered aromatic ring, optionally containing 1, 2 or 3 heteroatoms independently selected from N, O and S.

15. (canceled)

16. The method according to claim 1 wherein the compound of formula (I) is a compound of formula (Ia): 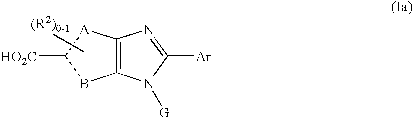
or a pharmaceutically acceptable salt thereof, wherein the dotted line represents a single or double bond.

17. The method according to claim 16 wherein in the compound of formula (Ia) A is C and B is S.

18. The method according to claim 17 wherein in the compound of formula (Ia) wherein R² is absent or R² is C₁₋₆ alkyl or aryl.

19. (canceled)

20. The method according to claim 18 wherein in the compound of formula (Ia) wherein G is hydrogen, C₃₋₈ cycloalkyl, C₃₋₈ cycloalkenyl or aryl.

21. (canceled)

22. The method according to claim 18 wherein in the compound of formula (Ia) Ar is a 6-membered ring containing 0, 1 or 2 N atoms.

23. (canceled)

24. The method according to claim 1 wherein the compound of formula (I) is a compound of formula (Ib): 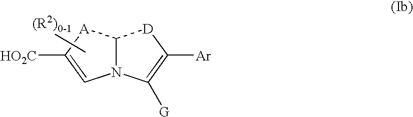
or a pharmaceutically acceptable salt thereof, wherein the dotted line represents a single or double bond.

25. The method according to claim 24 wherein in the compound of formula (Ib) A is S and D is N.

26. The method according to claim 25 wherein in the compound of formula (Ib) R² is absent or R² is C₁₋₆ alkyl or aryl.

27. (canceled)

28. (canceled)

29. The method according to claim 26 wherein in the compound of formula (Ib) G is hydrogen, C₃₋₈ cycloalkyl, C₃₋₈ cycloalkenyl or aryl.

30. The method according to claim 29 wherein in the compound of formula (Ib) Ar is a 6-membered ring containing 0, 1 or 2 N atoms.

31. (canceled)

32. The method according to claim 1 wherein the compound of formula (I) is selected from:
1-cyclohexyl-2-phenyl-1H-thieno[3,2-d]imidazole-5-carboxylic acid,
3-cyclohexyl-2-phenyl-3H-thieno[2,3-d]imidazole-5-carboxylic acid,
3-cyclohexyl-6-methyl-2-phenyl-3-thieno[2,3-d]imidazole-5-carboxylic acid,
3-cyclohexyl-2,6-diphenyl-3H-thieno[2,3-d]imidazole-5-carboxylic acid,
5,6-diphenylimidazo[2,1-b][1,3]thiazole-2-carboxylic acid,
6-phenylimidazo[2,1-b]thiazole-2-carboxylic acid,
5-cyclohex-1-en-1-yl-6-phenylimidazo[2,1-b][1,3]thiazole-2-carboxylic acid,
3-cyclohex-1-en-1-yl-2-phenylimidazo[2,1-b][1,3]thiazole-6-carboxylic acid;
and pharmaceutically acceptable salts thereof.

33. (canceled)

34. (canceled)

35. A pharmaceutical composition comprising a compound according to claim 37
or a pharmaceutically acceptable salt thereof, in association with a pharmaceutically acceptable carrier.

36. (canceled)

37. A compound selected from:
1-cyclohexyl-2-phenyl-1H-thieno[3,2-d]imidazole-5-carboxylic acid,
3-cyclohexyl-2-phenyl-3H-thieno[2,3-d]imidazole-5-carboxylic acid,
3-cyclohexyl-6-methyl-2-phenyl-3-thieno[2,3-d]imidazole-5-carboxylic acid,
3-cyclohexyl-2,6-diphenyl-3H-thieno[2,3-d]imidazole-5-carboxylic acid,
5,6-diphenylimidazo[2,1-b][1,3]thiazole-2-carboxylic acid,
6-phenylimidazo[2,1-b]thiazole-2-carboxylic acid,
5-cyclohex-1-en-1-yl-6-phenylimidazo[2,1-b][1,3]thiazole-2-carboxylic acid,
3-cyclohex-1-en-1-yl-2-phenylimidazo[2,1-b][1,3]thiazole-6-carboxylic acid;
and pharmaceutically acceptable salts thereof.
